(19) United States
(12) Patent Application Publication
(10) Pub. No.: US 2026/0124149 A1
(43) Pub. Date: May 7, 2026

(54) MODALITY, METHOD OF PRODUCING MODALITY, AND METHOD OF ADMINISTERING MODALITY INCLUDING POLYHYDRADED IONOGEN SOLUTION IN THE TREATMENT OF CHRONIC WOUNDS

(71) Applicant: Advanced Healing Technologies, LLC, Waco, TX (US)

(72) Inventors: Timothy Alcorn, Raleigh, NC (US); Anthony Gene Osha Gilstrap, Waco, TX (US); Claude Gene Gilstrap, Waco, TX (US); Berwin Singh Swami Vetha, Greenville, NC (US)

(21) Appl. No.: 19/380,413

(22) Filed: Nov. 5, 2025

Related U.S. Application Data
(60) Provisional application No. 63/716,533, filed on Nov. 5, 2024.

(51) Int. Cl. A61K 9/50; A61K 31/765; A61K 33/00; A61K 33/06; A61K 33/24; A61K 33/26; A61K 33/30; A61K 33/32; A61K 33/34

(52) U.S. Cl. CPC A61K 9/5031; A61K 31/765; A61K 33/00; A61K 33/06; A61K 33/24; A61K 33/26; A61K 33/30; A61K 33/32; A61K 33/34

(57) ABSTRACT

A treatment modality for use in the remediation of damaged tissues that involves protease down regulator solution impregnated microspheres, and the methods of production and therapeutic use thereof.

### CROSS-REFERENCE TO RELATED APPLICATION AND PRIORITY CLAIM

This application claims priority under 35 U.S.C. § 119(e) to U.S. Provisional Ser. No. 63/716,533 filed on Nov. 5, 2024 (Atty. Dkt. No ADVA 90-00002), which is hereby incorporated by reference in its entirety.

### BACKGROUND OF THE INVENTION

### Field of the Invention

The present disclosure relates to medicaments and treatment regimens in addressing chronic wounds in humans.

### Background of the Invention

Chronic wounds are a significant yet underappreciated health concern, impacting millions of individuals globally. Unlike acute wounds, which typically heal within a predictable timeframe, chronic wounds persist beyond three months and resist conventional treatments. These include diabetic foot ulcers, venous leg ulcers, and pressure ulcers, which are not only physically debilitating but also have severe psychological, social, and financial ramifications.

People suffering from chronic wounds experience a range of challenges that go beyond physical discomfort. The persistent pain, immobility, and risk of infection severely diminish their quality of life. Chronic wounds are often accompanied by complications such as cellulitis, osteomyelitis, and sepsis, which can lead to frequent hospitalizations or even amputations, especially in patients with diabetic ulcers.

Pain management is another significant challenge. Chronic wound pain can range from mild discomfort to severe, constant agony. Pain during dressing changes is particularly distressing, and patients often live in fear of these routines but necessary interventions. The psychological toll can be severe, leading to depression, anxiety, and social isolation as individuals struggle to cope with the long-term nature of their condition.

Treating chronic wounds places a heavy financial burden on both individuals and healthcare systems. In the United States alone, at the time of this writing, the estimated annual cost of chronic wound care ranges from $28 billion to $31 billion. This includes direct medical costs, such as hospital stays, physician services, surgical procedures, medications, wound dressings, and specialized therapies like hyperbaric oxygen treatment.

Key factors that drive up these costs include:
a. Long-term care: Chronic wounds require ongoing treatment and monitoring, often for years. Regular visits to wound care specialists, repeated debridement (removal of dead tissue), and daily dressing changes all add to the cost burden.
b. Complications and comorbidities: Chronic wounds are often complicated by underlying health conditions, such as diabetes, cardiovascular disease, and poor circulation, all of which increase the complexity and expense of treatment.
c. Advanced therapies: The use of advanced wound care products and technologies, like negative pressure wound therapy or bioengineered skin substitutes, while effective in some cases, can be costly and are not always covered by insurance.

The indirect costs associated with chronic wounds are also significant. Many individuals are unable to work or perform everyday activities due to their wounds, leading to loss of income, reduced productivity, and the need for long-term caregiving. In the case of severe disability, patients may need ongoing assistance with basic tasks, increasing the burden on family members or the need for professional home healthcare services.

The societal costs of chronic wounds extend beyond individual suffering and healthcare expenses, affecting public health systems and economies worldwide. Chronic wounds are particularly prevalent among aging populations, individuals with diabetes, and those with limited mobility, all of which are growing demographic in many countries.

The increasing prevalence of chronic wounds places a significant burden on healthcare systems, which must allocate substantial resources to wound management. Specialized wound care clinics, inpatient treatments, and the use of advanced wound care technologies require ongoing investment. Hospitals and healthcare professionals are faced with not only treating the wounds but also addressing the underlying conditions, which often complicate care.

Chronic wounds often lead to prolonged absenteeism from work, permanent disability, or early retirement, contributing to lost productivity. In particular, people with diabetic foot ulcers face a higher risk of lower extremity amputation, which severely limits their ability to contribute to the workforce. This, in turn, affects businesses and economies by reducing the number of active, able workers.

In cases where patients require long-term care due to chronic wounds and associated disabilities, the costs for nursing homes, assisted living facilities, and in-home caregiving can escalate. This puts additional pressure on families, insurance providers, and social welfare programs.

### SUMMARY OF THE INVENTION

In view of the foregoing, it would be advantageous to provide a new and more efficacious treatment regimen for chronic wounds than what is presently available to practitioners and their patients.

The present inventors here disclose a novel and unobvious medicament and delivery modality combination that addresses many shortcomings of presently available treatment regimens for chronic wound care.

### DETAILED DESCRIPTION OF THE PREFERRED EMBODIMENT

### Proteinase Down Regulatory (Custom Saline) and Its Role in Accelerating Tissue Repair

Proteinase Down Regulator which is a Custom Saline Solution, has demonstrated efficacy in accelerating tissue repair by downregulating matrix metalloproteinases (MMPs), specifically MMP-2 and MMP-9. These MMPs are implicated in various diseases and conditions, including delayed wound healing, respiratory damage from infections, and even cancer.

To date, Custom Saline Solution as used in wound care has been formulated into either a liquid or a gel containing Polyethylene Glycol (PEG), often impregnated into a bandage—a common formulation for wound healing compounds.

The present treatment or delivery modality of Custom Saline Solution as a remediating agent in wound care present several limitations:
a. Liquid Form: Effective delivery of active concentrations to the affected site is challenging, and the effectiveness is compromised by drying or absorption of exudates.
b. Impregnated Bandages: These cannot deliver effective concentrations to wounds with depth or irregular shapes.
c. Gels: Gels do not provide effective barriers to prevent wound contamination and cannot absorb exudates.
d. Non-Surface Tissues: Neither liquids nor gels can be effectively delivered to non-surface tissues.

The present inventors have discovered and verified application that using microspheres impregnated with Custom Saline Solution represents a significant advance in efficacy in treating wounds, even though the active agent of Custom Saline Solution is already known for use in wound care.

Microspheres can be applied to irregularly shaped wounds, providing an effective barrier and the ability to absorb exudates while keeping the wound moist.

Additionally, the size of the microspheres can be adjusted for use in nasal sprays, nebulizers, needles, etc., for delivery to non-surface tissues. Since the active ingredient is impregnated in the microsphere, it remains in contact with the affected tissue, unlike liquids that dissipate. The viscosity of gels prevents their use in most non-surface tissues.

### Processes for Producing Custom Saline-Impregnated Microspheres

The Custom Saline Solution component is formulated using a solution of water and citric acid (or other organic acids) with a low pH (approximately 2.5-3.5). In some formulations, the Custom Saline Solution component is soluble in water and releases free multivalent metal cations in quantities effective for treating or preventing viral infections in in-vitro tests. The multivalent metal cations include Zn²⁺, Rh⁺, Mg²⁺, Co²⁺, Ca²⁺, Cu²⁺, Fe²⁺, Fe³⁺, Ni²⁺, Ni³⁺, Al²⁺, Mn²⁺, Mn³⁺, Ti²⁺, Ti³⁺, Mo²⁺, and Mo³⁺.

The ions in the Custom Saline Solution formulation can be present in amounts ranging from about 0.01% to about 2% of the total weight of the composition. Specific ranges include about 0.01% to about 1%, about 0.02% to about 5%, or about 0.05% to about 0.5%. In some embodiments, the ionic multivalent metal component is present in amounts from about 0.00001% to about 5%, with specific ranges including about 0.00001% to about 0.0001%, about 0.0001% to about 0.001%, about 0.001% to about 0.01%, and about 0.1% to about 5%.

Some formulations may contain an ionic zinc component, present in amounts (in chloride salt equivalent) up to about 2% by weight of the composition. Specific ranges include less than about 5%, about 1%, about 0.01%, about 0.001%, about 0.0001%, about 0.00001%, or about 0.000001%.

An example formulation contains 1.93% w/w potassium citrate, 0.008% w/w rubidium chloride, 0.005% calcium chloride, and 0.0002% zinc chloride for a total of 1.945% of the total weight. Another example formulation contains 0.5% rubidium chloride, 0.03% of calcium chloride and 0.012% of zinc chloride for a total of 0.542% total. Another formulation contains 0.5% rubidium chloride, 0.03% of calcium chloride and 0.02% magnesium bromide for a total of 0.56% total. Another formulation contains 0.5% rubidium chloride, 0.03% of calcium chloride and 0.012% of zinc chloride, and 0.03% of manganese bromide for a total of 0.572% total, another formulation contains. Another formulation contains 1.93% w/w potassium citrate, 0.008% w/w rubidium chloride, 0.005% calcium chloride, and 0.0002% magnesium bromide for a total of 1.945% of the total weight. Another formulation contains 1.93% w/w potassium citrate, 0.008% w/w rubidium chloride, 0.005% calcium chloride, and 0.0002% manganese bromide for a total of 1.945% of the total weight. A final example formulation contains 0.00001% Fe²⁺, 0.0014% Zn²⁺, 0.0001% Ni²⁺, 0.001% Co, 0.008% Rh⁺, 0.0008% Ti²⁺, 0.005% Ca²⁺, and 2% K⁺ for a total of 2.02%.

Plastic microspheres are hollow and lightweight, made from synthetic polymers such as polymethyl methacrylate (PMMA), acrolein, glycidyl methacrylate, epoxy polymers, and hydroxypropyl methylcellulose (HPMC). Natural polymers include proteins (e.g., albumin, collagen, gelatin) and polysaccharides (e.g., chitosan, alginate, dextran). In a general medical use sense, the choice of microspheres depends on the intended use. In this case, recommended microspheres for practice of the present invention are believed to be optimally gelatin, collagen or dextran with diameters of 10 to 100M

The method of impregnating microspheres varies depending on the type used. Potential methods include:
a. Solvent Evaporation: A polymer-drug solution is dissolved in an organic solvent, which is then evaporated, leaving behind microspheres. More particularly, the process involves dissolving both the Custom Saline Solution and a biodegradable polymer (e.g., PLGA—Poly(lactic-co-glycolic acid)) in an organic solvent, such as dichloromethane or acetone. This solution is then emulsified into an aqueous phase containing a surfactant or stabilizer. After forming droplets (which become microspheres), the organic solvent evaporates, leaving behind hardened microspheres encapsulating the Custom Saline.
b. Solvent Diffusion: Similar to solvent evaporation, but the Custom Saline Solution diffuses into a nonsolvent (aqueous phase) to form microspheres. More particularly, this technique is used to encapsulate drugs with hydrophilic or hydrophobic properties. It involves preparing an oil-in-water or water-in-oil emulsion in which the Custom Saline Solution and polymer are dissolved in an organic solvent. After forming an emulsion, the solvent diffuses into the aqueous phase, leading to the formation of solid microspheres.
c. Spray Dying: This is a process commonly used for heat-sensitive drugs and proteins. In this method, the Custom Saline Solution and polymer are dissolved or suspended in a solvent, and the solution is atomized into a fine spray using a spray dryer. As the solvent evaporates in the drying chamber, solid microspheres are formed.
d. The first of several chemical methods—Ionic Gelation—involves crosslinking polymers using ions (e.g., calcium ions) to form microspheres.
e. Emulsion Solvent Evaporation: this process involves the creation of an emulsion (typically oil-in-water) where a polymer and drug are dissolved in an organic solvent that is immiscible with water. The organic solvent is then evaporated, leaving behind solid microspheres containing the Custom Saline.
f. Coacervation: Polymers are precipitated from a solution by adding a nonsolvent, resulting in microspheres. This method is used to produce microspheres by inducing phase separation of a polymer from its solution. A polymer is dissolved in a suitable solvent, and the Custom Saline Solution is dispersed in this solution. Phase separation (coacervation) is triggered by adding a nonsolvent, changing temperature, or adjusting pH. The polymer precipitates around the drug particles, forming microspheres.

Custom Saline-impregnated microspheres, however they are produced, are suspended in a gel or liquid suspension, optimally with a density of 10-100 mg /l depending on Custom Saline Solution concentration and microsphere side.

### Exemplary Treatment Regimens

For a non-healing wound, such as a pressure ulcer, a treatment regimen using Custom Saline-impregnated microspheres having Custom Saline Solution concentrations in the lower end of the ranges described above would optimally involve application to completely cover the affected area with depth of 3-5 mm, to be applied 1× daily for 45 days or until closure. For such treatment involving the use of Custom Saline Solution concentrations in the upper end of the ranges described above would optimally involve application to completely cover the affected area with a thickness of 3-5 mm, to be applied 1× daily for 20 days or until closure.

Experimental and confidential applications of Custom Saline-impregnated microspheres in the treatment of chronic wounds have produced promising results. For example in animal studies Custom Saline Solution microspheres are able to demonstrate complete wound closure in 90% of subjects within 20 days. In additional studies in health human subjects, the impregnated beads decreased the time to healing by 50% on clean lacerations. In additional clinical studies of deep diabetic ulcers, the Custom Saline Solution microspheres demonstrated the level of efficacy (>75%) as the impregnated bandages on shallow ulcers. Overall, the Custom Saline Solution microspheres has shown superior efficacy (% wound closure and closure time) for both acute wounds or chronic wounds when compared to over impregnated bandages including hydrogel bandages, bandages that containing sliver or other antimicrobial agents.

Although the invention has been described with reference to specific embodiments, this description is not meant to be construed in a limited sense. Various modifications of the disclosed embodiments, as well as alternative embodiments of the inventions will become apparent to a person skilled in the art upon the reference to the description of the invention. It is, therefore, contemplated that the appended claims will cover such modifications that fall within the scope of the invention.

1. A composition for use in the remediation of damaged tissues within a chronic wound comprising:
a proteinase down regulator solution for accelerating tissue repair of the damaged tissues within the chronic wound comprising a proteinase down regulator, said proteinase down regulator solution being encapsulated in one or more microspheres, wherein the one or more microspheres comprise a polymer for encapsulating the proteinase down regulator solution.

2. The composition of claim 1 wherein said one or more microspheres are fabricated inclusive of constituents selected from a group consisting of Poly(lactic-co-glycolic acid) (PLGA), Polylactic acid (PLA), Polyanhydrides, and Polyorthoesters.

3. The composition of claim 1 wherein said one or more microspheres are fabricated inclusive of constituents selected from a group consisting of albumin, gelatin, Chitosan and Alginate.

4. The composition of claim 1 further comprising, a bandage into which said microsphere are incorporated embedded to enable application of the one or more microspheres to the damaged tissues within the chronic wound.

5. The composition of claim 1 further comprising a solution a constituent of which is said one or more microspheres, for application of said one or more microsphere to the damaged tissues, with said solution residing within an aerosol dosage delivery device.

6. The composition of claim 1 wherein said one or more microspheres are fabricated to encapsulate proteinase down regulator constituents selected from a group consisting of Zn²⁺, Rh⁺, Mg²⁺, Co²⁺, Ca²⁺, Cu²⁺, Fe²⁺, Fe³⁺, Ni²⁺, Ni³⁺, Al²⁺, Mn²⁺, Mn³⁺, Ti²⁺, Ti³⁺, Mo²⁺, and Mo³⁺.

7. The composition of claim 1 wherein said microspheres encapsulate proteinase down regulator constituents selected from a group consisting of Br²⁺, Rh⁺, Mg²⁺, Co²⁺, Ca²⁺, Cu²⁺, Fe²⁺, Fe³⁺, Ni²⁺, Ni³⁺, Al²⁺, Mn²⁺, Mn³⁺, Ti²⁺, Ti³⁺, Mo²⁺, and Mo³⁺.

8. The composition of claim 7 further comprising as a constituent of said proteinase down regulator poly-ethylene glycol.

9. (canceled)

10. A composition for use in the remediation of damaged tissues within a chronic wound comprising:
a proteinase down regulator solution comprising water and an organic acid for accelerating tissue repair of the damaged tissues within the chronic wound, said proteinase down regulator solution being encapsulated in one or more microspheres, wherein the one or more microspheres comprise a polymer for encapsulating the proteinase down regulator solution.

11. The composition of claim 10 wherein said one or more microspheres are fabricated to encapsulate proteinase down regulator constituents selected from a group consisting of Zn²⁺, Rh⁺, Mg²⁺, Co²⁺, Ca²⁺, Cu²⁺, Fe²⁺, Fe³⁺, Ni²⁺, Ni³⁺, Al²⁺, Mn²⁺, Mn³⁺, Ti²⁺, Ti³⁺, Mo²⁺, and Mo³⁺.

12. The composition of claim 10 wherein said one or more microspheres are fabricated to encapsulate proteinase down regulator constituents selected from a group consisting Br²⁺, Rh⁺, Mg²⁺, Co²⁺, Ca²⁺, Cu²⁺, Fe²⁺, Fe³⁺, Ni²⁺, Ni³⁺, Al²⁺, Mn²⁺, Mn³⁺, Ti²⁺, Ti³⁺, Mo²⁺, and Mo³⁺.

13. A method for treating damaged tissues within a chronic wound comprising the steps of:
selecting a proteinase down regulator solution for accelerating tissue repair of the damaged tissues within the chronic wound comprising a proteinase down regulator encapsulated within one or more microspheres, wherein the one or more microspheres comprise a polymer for encapsulating the proteinase down regulator solution; and
applying said one or more microspheres having the encapsulated proteinase down regulator solution therein to said damaged tissue within the chronic wound.

14. The method of claim 13 wherein said one or more microspheres are fabricated inclusive of constituents selected from a group consisting of Poly(lactic-co-glycolic acid) (PLGA), Polylactic acid (PLA), Polyanhydrides, and Polyorthoesters.

15. The method of claim 13 wherein the step of applying said one or more microspheres having the encapsulated proteinase down regulator solution further comprises applying said one or more microspheres through use of a bandage having been impregnated with said one or more microspheres having the encapsulated proteinase down regulator solution therein.

16. The method of claim 13 wherein the step of applying said one or more microspheres having the encapsulated proteinase down regulator solution further comprises applying said one or more microspheres through use of an aerosol dosage delivery device in which said one or more microspheres having the encapsulated proteinase down regulator solution therein has been placed for later administration.

17. The method of claim 13 wherein said one or more microspheres are fabricated inclusive of one or more proteinase down regulator constituents selected from a group consisting of Zn²⁺, Rh⁺, Mg²⁺, Co²⁺, Ca²⁺, Cu²⁺, Fe²⁺, Fe³⁺, Ni²⁺, Ni³⁺, Al²⁺, Mn²⁺, Mn³⁺, Ti²⁺, Ti³⁺, Mo²⁺, and Mo³⁺.

18. The method of claim 13 wherein said one or more microspheres are fabricated inclusive of one or more proteinase down regulator constituents selected from a group consisting of Br²⁺, Rh⁺, Mg²⁺, Co²⁺, Ca²⁺, Cu²⁺, Fe²⁺, Fe³⁺, Ni²⁺, Ni³⁺, Al²⁺, Mn²⁺, Mn³⁺, Ti²⁺, Ti³⁺, Mo²⁺, and Mo³⁺.

19. The method of claim 13 wherein a constituent of said proteinase down regulator is poly-ethylene glycol.

20. A method for producing a treatment modality for treatment of damaged tissues within chronic wound comprising the steps of:
producing a proteinase down regulator solution for accelerating tissue repair of the damaged tissues within the chronic wound comprising a custom saline solution acting as a proteinase down regulator;
producing one or more microspheres comprising a polymer; and
encapsulating portions of the custom saline solution into the one or more microspheres.

21. The method of claim 20 wherein producing said one or more microspheres further comprises fabricating the one or more microspheres inclusive of constituents selected from a group consisting of Poly(lactic-co-glycolic acid) (PLGA), Polylactic acid (PLA), Polyanhydrides, and Polyorthoesters.

22. The method of claim 20 wherein producing said one or more microspheres further comprises fabricating the one or more microspheres inclusive of constituents selected from a group consisting of albumin, gelatin, Chitosan and Alginate.

23. The method of claim 20 wherein further comprising the steps of:
selecting a bandage; and
at least partially impregnating said bandage with one or more of said proteinase down regulator solution impregnated microspheres.

24. The method of claim 20 further comprising, generating a solution a constituent of which is one or more of said microspheres, and locating the solution within an aerosol dosage delivery device for application of said one or more microsphere to damaged tissues within the chronic wound.

25. The method of claim 20 wherein producing said proteinase down regulator further comprises selecting the proteinase down regulator constituents from a group consisting of Zn²⁺, Rh⁺, Mg²⁺, Co²⁺, Ca²⁺, Cu²⁺, Fe²⁺, Fe³⁺, Ni²⁺, Ni³⁺, Al²⁺, Mn²⁺, Mn³⁺, Ti²⁺, Ti³⁺, Mo²⁺, and Mo³⁺.

26. The method of claim 20 wherein producing said proteinase down regulator further comprises selecting the proteinase down regulator constituents selected from a group consisting of Br²⁺, Rh⁺, Mg²⁺, Co²⁺, Ca²⁺, Cu²⁺, Fe²⁺, Fe³⁺, Ni²⁺, Ni³⁺, Al²⁺, Mn²⁺, Mn³⁺, Ti²⁺, Ti³⁺, Mo²⁺, and Mo³⁺.

27. The invention method of claim 20 wherein producing said proteinase down regulator further comprises selecting as a constituent of said proteinase down regulator poly-ethylene glycol.

28. The composition of claim 1, wherein the proteinase down regulator solution further comprise a custom saline solution including citric acid and having a pH in a range of approximately 2.5-3.5.
